(19) United States
(12) Patent Application Publication
(10) Pub. No.: US 2004/0047815 A1
(43) Pub. Date: Mar. 11, 2004

(54) Dentifrice product

(76) Inventors: Ana Torrado Bonals, Barcelona (ES); Manuel Valiente Malmagro, Barcelona (ES)

Correspondence Address: STEINBERG & RASKIN, P.C., 1140 AVENUE OF THE AMERICAS, 15th FLOOR, NEW YORK, NY, 10036-5803, US

(21) Appl. No.: 10/451,352

(22) PCT Filed: Dec. 20, 2001
(86) PCT No.: PCT/IB01/02709

(51) Int. Cl. A61K 7/18

(52) U.S. Cl. 424/52

(57) ABSTRACT

Comprises a surfactant a cellulose derivative, additives and at least one resin charged with Ca⁺², F⁻, PO₄³ oZn⁺² cations or anions, with the calcium, fluoride and phosphate ions in a molar ratio of 2:1:1 and the zinc ions in dry-weight proportion with respect to the resin or resins of between 0.5 and 2%. It is used as a dental-tissue remineralising agent or for making a medicine for the treatment of buccal diseases and, in particular, for the treatment of caries.

### FIELD OF THE INVENTION

This invention relates to a dentifrice product which includes a resin encapsulated within a matrix through which ions are released.

This invention also relates to the use of said product in remineralisation of dental tissues.

### BACKGROUND OF THE INVENTION

This invention relates to a dentifrice product with remineralising effect.

known in the art are dentifrices which include a surfactant, a cellulose derivative and additives. Unknown in the state of the art are toothpastes which incorporate ion-exchange resins encapsulated within a matrix. In some of the known products the ion-exchange process is used in order to synthesise components which act as bactericidal agents (U.S. Pat. No. 4,621,120, 1986), dental caries inhibitors (DE patent 3605656, 1987), components responsible for the properties of the toothpaste: its abrasiveness (Hertzenberg, E. P. et al., Stud. Surf. Sci. Catal., 24:589-596 (1985)), its irritability (U.S. Pat. No. 4,391,798, 1983), etc. Others include in their formulation zeolite-type inorganic materials (U.S. Pat. No. 4,349,533, 1982, EP patent 297563, 1989).

There exist in the state of the art numerous studies of remineralisation, with characteristic appearance of calcium, fluoride and phosphate ions in the form of various inorganic salts. Some cases are known in which the pastes include only anionic components, whether fluoride alone (Mellberg, J. R. et al., Caries Res., 25:65-69 (1991)) or a fluoride and phosphate mixture (U.S. Pat. No. 4177258, 1979). This type of compositions give rise to the formation of insoluble compounds by bringing the different ions into contact, since a significant fraction of the paste formulation is water, so that there is a reduction of the effective quantity of ions available for carrying out remineralisation. Also known is the use of separate compartments, one for the cationic components and another for the anionic (U.S. Pat. No. 5,858,333, 1999), so that the insoluble precipitate forms when the two parts are placed in contact, and is then deposited directly upon the damaged site, so that the remineralising power of the paste increases.

Known in the state of the art is a method which involves two stages (Magas, S. et al., Czas. Stomatol., 43(6):323-327 (1990)); a first stage based on treatment with a paste which includes anionic components (phosphate, monofluorophosphate and fluoride salts) and a second stage based on treatment with a paste which includes calcium salts and other cations.

Studies on remineralisation can be carried out by means of three different techniques:

In vitro (Cheng, C. et al., Beijing Yike Daxue Xuebao, 23(4):305-307 (1991), Marinelli, C. B. et al., Caries Res., 31(6):418-422 (1997)) with extracted teeth.

In situ (Mellberg, J. R. et al., Caries Res., 25:65-69 (1991)) using extracted, damaged teeth in the natural biological medium.

In vivo (De Kloet, H. et al., J. Dent. Res., 65(12):1410-1414 (1986), Mellberg, J. R. et al., J. Dent. Res., 65(8):1078-1083 (1986)) with non-extracted teeth.

Recently there have appeared studies in which attempts are made to develop products which in addition to achieving remineralisation improve characteristics such as whitening and the removal of stains (WO patent 2000047173, 2000).

### DESCRIPTION OF THE INVENTION

In this invention the effect of encapsulation of resins on the speed of release of ions in toothpaste matrices was determined. Thus, a study was made of the effect of the surfactant and of the cellulose derivative on said speed.

The dentifrice product of this invention is characterised in that it comprises at least one ion-exchange resin charged with cations or anions, said resin or resins comprising Ca⁺², F⁻, PO₄⁻³ o Zn⁺² ions. Preferably, it comprises a cellulose derivative which has no calcium ion sequestering agents and, more preferably still, a non-ionic surfactant. When the calcium, fluoride and phosphate ions are present in the mixture of resins, they are preferably in a molar ratio of 2:1:1. The Zn⁺² ions included in the resin or resins are in a dry-weight proportion between 0.5-2%.

Said resin or mixture of resins comprises:

Cationic resin of the weak acid or strong acid character charged with calcium ions (R—Ca).

Cationic resin of the weak acid or strong acid character charged with zinc ions (R—Zn).

Anionic resin of the weak base or strong base character charged with phosphate ions (R—F)

Anionic resin of the weak base or strong base character charged with fluoride ions (R—PO4).

In the present invention, the term “strong acid/base” is referred to an acid or a base that it is totally dissociated in an aqueous media.

In the same way, in the present invention, the term “weak acid/base” is referred to an acid or a base that it is partially dissociated in an aqueous media.

More preferably still, the toothpaste composition comprises a proportion by weight of the resin or mixture or resins between 1-15%.

Table 1 shows the main components of the different 5 toothpaste bases used.

TABLE 1
Main composition of the various toothpaste bases.

| | Base | Base | Base | Base | Base |
|---|---|---|---|---|---|
| | 1 | 2 | 3 | 4 | 5 |
| Sodium laurylsulphate | ✓ | — | ✓ | — | — |
| Potyoxyl 40 of | — | — | — | — | ✓ |
| hydrogenated castor oil | | | | | |
| Sodium | ✓ | ✓ | — | — | — |
| carboxymethylcellulose | | | | | |
| Methylcellulose MP424C | — | — | ✓ | ✓ | ✓ |
| Anhydrous colloidal | ✓ | ✓ | ✓ | ✓ | ✓ |
| silica | | | | | |
| Glycerine | ✓ | ✓ | ✓ | ✓ | ✓ |
| Propylene glycol | ✓ | ✓ | ✓ | ✓ | ✓ |
| Water | ✓ | ✓ | ✓ | ✓ | ✓ |

These bases were used to prepare different toothpaste samples containing the concentrations of 10 monocomponent and NMTD stated below:

TABLE 2
Concentration of monocomponent and NMTD in the
different samples of toothpaste expressed in percentage by weight.

| | | | | | % NMTD |
|---|---|---|---|---|---|
| | % Monocomponent | | | | R-Ca²⁺ + |
| | R-Ca²⁺ | R-Zn²⁺ | R-F⁻ | R-PO₄³⁻ | R-Zn²⁺ + R-F + R-PO₄³⁻ |
| Base | 10 | 10 | 10 | 10 | — |
| 1 | | | | | |
| Base | 10 | — | — | — | — |
| 2 | | | | | |
| Base | 10 | — | — | — | — |
| 3 | | | | | |
| Base | 10 | — | — | — | — |
| 4 | | | | | |
| Base | 10 | — | 10 | — | 10/15 |
| 5 | | | | | |

Finally, an evaluation was carried out of the effect on release of ions the incorporation of some of the additives most widely used in conventional toothpaste formulations. This is the case of the titanium oxide (TiO₂) typically used and for whitening the paste for subsequent addition of the desired colorant. The work was carried out with samples of base paste 5 containing TiO₂ and 10% of NMTD.

Preferably, the cellulose derivatives used in the invention are selected from among methylcellulose; hydroxyethylcellulose; hydroxypropylcellulose or hydroxypropylmethylcellulose.

Preferably, the non-ionic surfactant is selected from among polyoxyethylenesorbitan monolaureate; polyoxyethylenesorbitan monopalmitate; monostearate; polyoxyethylenesorbitan mono-oleate; polyoxyethylenesorbitan trioleate; polyoxyethylene stearate; polyoxyethylene laureate; polyoxyl 40 of hydrogenated castor oil; polyoxyl 35 or 60 of castor oil and polyoxyethylenated ricin oil.

In parallel to this an “in vitro” study was also made of the remineralising action of the NMTD product incorporated, in a proportion of 10 and 15% respectively, into toothpaste base 5.

The results obtained from the study of the samples made up of base 1+10% of monocomponent have showed that the release of the anions in the presence of toothpaste is carried out more quickly. In the case of the cations, on the other hand, the effect is entirely the contrary. It was also found that the ions of interest were not present in the toothpaste base matrix (white). Both observations on the difference observed in the release of cations and anions compared with the respective pure components are attributed to the presence of ionic surfactants (sodium laurylsulphate) and to compounds with residual ionic groups in the base of the paste (carboxymethylcellulose).

Thus, with the intention of optimising release of all the ions of the respective monocomponents-toothpaste new modified toothpaste matrices were prepared in order to carry out a study of the components responsible for the low release of the cations. Calcium was studied preferably, as zinc is a minority component in the mixture which constitutes the final product.

From the study of the speed of release of the Ca²⁺ion of the samples: base 2+10% Ca-monocomponent, base 3+10% Ca-monocomponent and base 4+10% Ca-monocomponent it was observed that the release of the Ca²⁺ ion of the different toothpaste matrices was clearly influenced both by the surfactant and by the carboxymethylcellulose. However, the effect exercised by this last was greater than that of the surfactant, and the sample without either of these components was the one which presented a release of the ion equivalent to the behaviour observed in the resin system in the absence of toothpaste matrix. (See FIG. 1 attached)

The kinetic curves of release of the Ca²⁺ and F⁻ ions of the sample: base 5+10% of the respective monocomponent show that the release of both cations and anions was carried out at practically the same speed as with the respective components without toothpaste.

Thus, the study of release of the ions of sample base 5+10% of NMTD led to the conclusion that there are no significant differences between release of the ions in the mixture of resins in the presence of this toothpaste matrix and that which takes place in the absence of said paste. (See FIG. 2 enclosed) The study on evaluation of the effect exercised by the concentration of the active ingredient (NMTD) in the samples of toothpaste base of type 5 on release of the ions again indicates that the latter are released at a speed entirely comparable to that obtained with the active ingredient in the absence of toothpaste matrix.

The results of release from the sample of paste bases 5 containing TiO₂ and 10% NMTD showed no significant differences between release of the ions in encapsulated and non-encapsulated NMTD.

In accordance with the results obtained, the presence of carboxymethylcellulose and sodium laurylsulphate in the toothpaste base causes an acceleration in the speed of release of the anions while it delays release of the cations. This fact is due, on the one hand, to the anion-displacing behaviour exercised by said components of the toothpaste and, on the other hand, to the interaction (complexing) between those components and the Ca²⁺ and Zn²⁺ ions contributing to delaying their release.

The optimisation of the toothpaste base by replacement of the carboxymethylcellulose by methylcellulose and of the ionic surfactant by a non-ionic surfactant permits a toothpaste to be obtained that is compatible with release of all the ions forming part of the NMTD material responsible for remineralisation of the dental tissues.

Release of the calcium, fluoride and phosphate ions is not affected by the quantity of active ingredient introduced into the toothpaste matrix, nor by the presence of additives such as the whitening agent, titanium oxide.

The results obtained from the “in vitro” study of paste base 5 containing 10 or 15% of NMTD product showed that the greatest obliteration of dentinary tubules, in comparison with all the materials used to carry out said study, was achieved with the toothpaste containing 15% NMTD (See FIG. 3 enclosed). Moreover, the toothpastes which contain the NMTD product have a greater affinity with the dentin than the other products studied, thus revealing that the NMTD material can maintain its capacity for acting in spite of being encapsulated in a toothpaste matrix.

The dentifrice product described in this invention, which comprises at least one ion-exchange resin can be used in remineralisation of dental tissues. Due to this effect, on the zone of the tooth in contact with the toothpaste, this material is also beneficial for the preventive or symptomatic treatment of buccal infections such as caries and gingivitis.

### Figures

FIG. 1: shows evaluation of the effect of the surfactant and cellulose derivatives on the speed of release of the calcium ion in the different toothpaste matrixes.

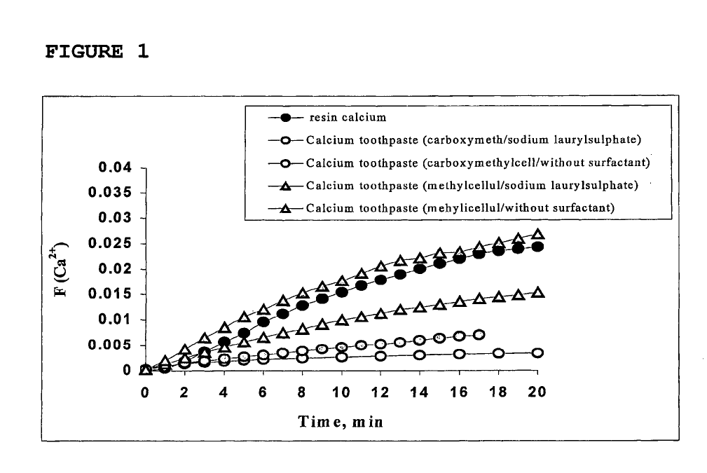

FIG. 2: shows the degree of conversion of the calcium ion of the base component 5+10% NMTD (PLS system, discontinuously and at 37° C.).

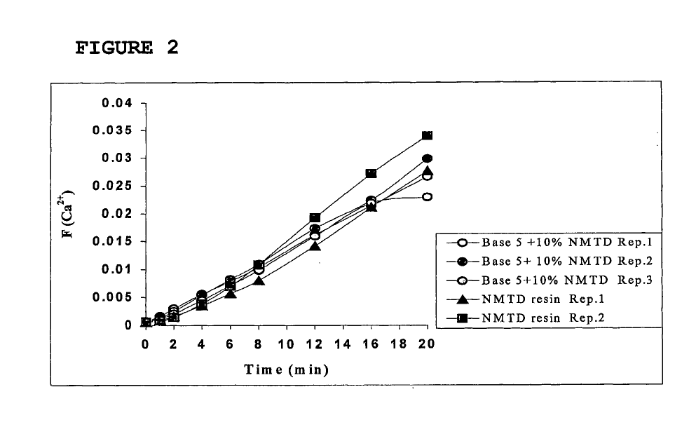

FIG. 3: shows the percentage of dentinary tubules obliterated.

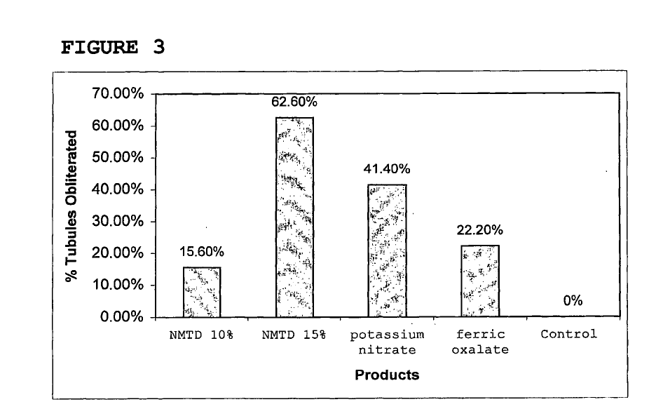

1. Dentifrice product, which comprises a surfactant, a cellulose derivative and additives, characterised in that it includes at least one resin charged with cations or anions, said resin or resins comprising the Ca⁺², F⁻, PO₄⁻³ o Zn⁺² ions.

2. Dentifrice product as claimed in claim 1 wherein the cellulose derivative has no calcium ion sequestering groups and the surfactant is non-ionic.

3. Dentifrice product, as claimed in claim 1, wherein the resin or resins comprise calcium, fluoride and phosphate ions in a molar ratio of 2:1:1.

4. Dentifrice product as claimed in claim 1 wherein the resin or mixture of resins comprises:
Cationic resin of the weak acid or strong acid type charged with calcium ions (R—Ca).
Cationic resin of the weak acid or strong acid type charged with zinc ions (R—Zn).
Anionic resin of the weak base or strong base type charged with phosphate ions (R—F)
Anionic resin of the weak base or strong base type charged with fluoride ions (R—PO4).

5. Dentifrice product, as claimed in claim 1, wherein the mixture of resins is in a proportion by weight of between 1-15%.

6. Dentifrice product, as claimed in claim 1, wherein the Zn⁺² ion is in a dry-weight proportion with the respect to the resin of between 0.5-2%

7. Dentifrice product, as claimed in claim 1, wherein said cellulose derivatives are selected from among methylcellulose; hydroxyethylcellulose; hydroxypropylcellulose or hydroxypropylmethylcelluose.

8. Dentifrice product, as claimed in claim 1, wherein said non-ionic surfactant is selected from among polyoxyethylenesorbitan monolaureate; polyoxyethylenesorbitan monopalmitate; monostearate; polyoxyethylenesorbitan mono-oleate; polyoxyethylenesorbitan trioleate; polyoxyethylene stearate; polyoxyethylene laureate; polyoxyl 40 of hydrogenated castor oil; polyoxyl 35 or 60 of castor oil and polyoxyethylenated ricin oil.

9. Dentifrice product, as claimed in any of the previous claims, for use thereof in remineralisation of dental tissues.

10. Use of a dentifrice product, as claimed in any of the previous claims, for the manufacturing of a medicine for the treatment of buccal infections.

11. Use of a dentifrice product, as claimed in any of the previous claims, for the manufacturing of a medicine for the treatment of caries.
